(19) United States
(12) Plant Patent Application Publication
(10) Pub. No.: US 2016/0286708 P1
(43) Pub. Date: Sep. 29, 2016

(54) Salvia plant named 'DSALDPOCN'

(50) ;

(71) Applicant: Gavriel Danziger, Moshav Mishmar Hashiva (IL)

(72) Inventor: Gavriel Danziger, Moshav Mishmar Hashiva (IL)

(21) Appl. No.: 14/545,104

(22) Filed: Mar. 27, 2015

(51) Int. Cl. A01H 5/00

(52) U.S. Cl. PLT/475

(57) ABSTRACT

A new and distinct Salvia cultivar named ‘DSALDPOCN’ is disclosed, characterized by early flowering of deep blue flowers, very full and mounded habit. Additionally all the flowers are at the same height. The new variety is a Salvia, normally produced as an outdoor garden or container plant.

### LATIN NAME OF THE GENUS AND SPECIES

Salvia farinacea

### VARIETY DENOMINATION

‘DSALDPOCN’

### BACKGROUND OF THE INVENTION

The new Salvia cultivar is the product of a planned breeding program conducted by the inventor, Gavriel Danziger, in Moshav Mishmar Hashiva, Israel. The objective of the breeding program was to produce new Salvia varieties for ornamental commercial applications. The open pollination resulting in this new variety was made during 2010.

The seed parent is the, unpatented, proprietary variety referred to as Salvia ‘SAF-9-971’. The pollen parent is unknown as it was an open pollination breeding program. The new variety was discovered in June of 2011 by the inventor in a group of seedlings resulting from the 2010 open pollination, in a research greenhouse in Moshav Mishmar Hashiva, Israel.

Asexual reproduction of the new cultivar was performed by vegetative apical cuttings. This was first performed at a research greenhouse in Moshav Mishmar Hashiva, Israel in July of 2011 and has shown that the unique features of this cultivar are stable and reproduced true to type in multiple successive generations.

### SUMMARY OF THE INVENTION

The cultivar ‘DSALDPOCN’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘DSALDPOCN’ These characteristics in combination distinguish ‘DSALDPOCN’ as a new and distinct Salvia cultivar:

1. Very full and mounded plant habit.

2. Early flowering.

3. Deep blue flower color.

4. Flowers occurring at the same height.

Plants of the new cultivar ‘DSALDPOCN’ are similar to plants of the seed parent, Salvia ‘SAF-9-971’ in most horticultural characteristics, however, plants of the new cultivar ‘DSALDPOCN’ differ in the following;

1. Deep blue flowers. The seed parent has white flowers.

2. The new variety blooms earlier.

3. The new variety produces more flowers.

4. Shorter peduncles.

### COMMERCIAL COMPARISON

Plants of the new cultivar ‘DSALDPOCN’ are comparable to the unpatented commercial variety Salvia ‘SALLYFUN BLUE EMOTION’. The two Salvia varieties are similar in most horticultural characteristics; however, the new variety ‘DSALDPOCN’ differs in the following:

1. The new variety has a more compact plant habit.

2. The new variety has a more full plant habit.

3. The new variety does not have noticeable white eyes on the flower, as the comparator.

4. The new variety has shorter peduncles.

Plants of the new cultivar ‘DSALDPOCN’ can also be comparable to the unpatented commercial variety Salvia ‘SALLYFUN BICOLOR BEAUTY’. The two Salvia varieties are similar in most horticultural characteristics; however, the new variety ‘DSALDPOCN’ differs in the following:

1. The new variety has lower petal and upper petals the same shade of blue, whereas comparator has 2 different colors—the lower petal in shades of light blue and the upper in darker blue.

2. More compact and mounded habit. The comparator habit is more spreading.

### BRIEF DESCRIPTION OF THE PHOTOGRAPHS

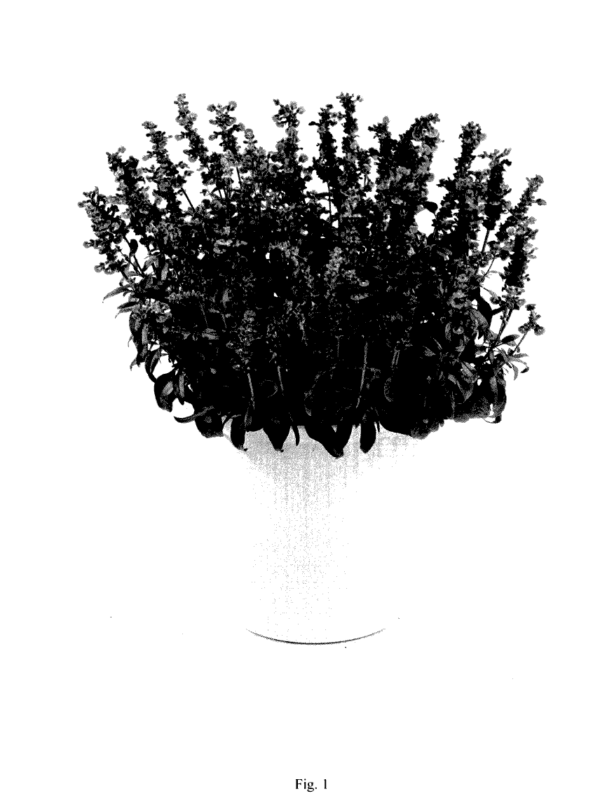

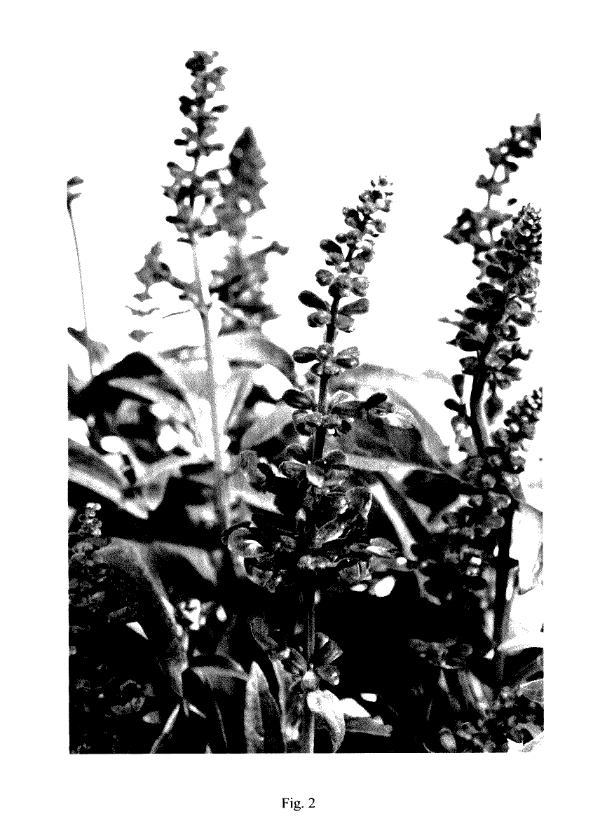

The accompanying photograph in FIG. 1 illustrates in full color a typical plant of ‘DSALDPOCN’ grown in a greenhouse, in a 13 cm pot. Age of the plant photographed is approximately 15 weeks from a rooted cutting. FIG. 2 illustrates a close up of the flowers and foliage. The photographs were taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Society Mini Colour Chart 2001 except where general terms of ordinary dictionary significance are used. The following observations and measurements describe ‘DSALDPOCN’ plants grown in the Autumn, to Spring in moderate climate in a greenhouse, in Moshav Mishmar Hashiva, Israel. The growing temperature ranged from 20° C. to 30° C. during the day and from 16° C. to 25° C. during the night. General light conditions are bright, normal sunlight. Measurements and numerical values represent averages of typical plant types.
Botanical classification: Salvia farinacea ‘DSALDPOCN’
Propagation:
   Type of propagation typically used.—vegetative cutting.
   Time to initiate roots.—5-7 days at approximately 25° C.
   Time to produce a rooted cutting.—About 10-14 days at 25° C.
Plant:
   Age of plant described.—Approximately 15 weeks.
   Container size of the plant described.—13 cm.
   Overall plant shape or growth habit.—Mounded.
   Length of primary lateral branches.—Approximately 30 cm.
   Diameter of lateral branches.—Approximately 0.6 cm.
   Quantity of lateral branches.—About 20-25.
   Branch color.—Yellow-green group 144A RHS.
   Branch texture/pubescence.—Pulverulent, slightly ribbed longwise.
   Internode length.—Approximate average 2 cm.
Foliage:
   Leaf.—Arrangement: Opposite Quantity: Approximately 20 per main branch. Average Length: Approximately 8 cm. Average Width: Approximately 3 cm. Shape of blade: Ovate Apex: Broadly acute Base: Pointed Attachment: Petiolate Margin: Very slightly crenate Texture of top surface: Smooth, slightly glossy, sporadically canescent. Texture of bottom surface: Smooth, matte, sporadically canescent. Color: Young foliage upper side: Near Yellow-green 146B RHS Young foliage under side: Near Yellow-green 146A RHS Mature foliage upper side: Near Yellow-green 147A RHS Mature foliage under side: Near Yellow-green 147A RHS Venation: Type: Pinnate Venation color upper side: Near Yellow-green group 146B RHS Venation color under side: Near Yellow-green group 146C RHS Petiole: Average Length: Approximately 3.5 cm. Diameter: Approximately 0.2 cm. Color: Near Yellow-green group 144B RHS.
Flower:
   Natural flowering season.—From early spring to late autumn in moderate climate.
   Inflorescence type and habit.—Bilabiate flowers arranged in vertical clusters on spike, upright.
   Flower longevity on plant.—about 3 to 5 days, depending on temperature.
   Quantity of flowers.—About 10-15 blooming spikes per plant.
   Inflorescence size.—Diameter: Approximately 3.5 cm. Height: Approximately 18 cm. Flowers and buds per inflorescence: Average range 60 to 90.
   Peduncle.—Length: 15-18 cm. Diameter: 0.5 cm Texture: Pulverulent, slightly ribbed longwise Color: Near Violet-Blue group N92C RHS Orientation: About 30° from vertical Strength: Strong Petal arrangement: The corolla is sympetalous and typically bilabiate with 2 small, highly fused lobes forming an upper lip and 3 larger highly fused lobes forming a lower lip.
   Individual flowers.—Size: Upper lip (tube like) Diameter: Approximately 0.3 cm. Length: Approximately 0.5 cm. Lower lip (4 fused lobe) Length: Approximately 1 cm. Width: Approximately 1.2 cm. Persistence: Non-persistent. Fragrance: Very sweet, nectar-like fragrance.
   Petals.—One upper lip and one lower lip with 4 lobes. Lips are fused at base. Upper lip has smooth short feather like hairs, lower lip is smooth and hairless.
   Margin.—Slightly wavy Tip shape: Upper lip: Rounded Lower lip: Blunt Length: Upper lip: 0.5 cm Lower lip: 1 cm Tube: 0.5 cm Width: Upper lip: 0.3 cm Lower lip: 1 cm Tube: 0.3 cm Texture: Upper lip: Upper surface: Smooth, short feather like hairs; Under surface: Glabrous Lower lip: Upper surface: Glabrous; under surface: Glabrous. Tube: Upper surface: Glabrous; under surface: Glabrous.
   Color.—Upper Lip: When opening: Inner surface: Near Violet-Blue group 93A RHS Outer surface: Near Violet-Blue group N92B RHS Fully opened: Inner surface: Near Violet-Blue group 93A RHS Outer surface: Near Violet-Blue group 93A RHS Fading: Inner surface: Near Violet-Blue group 93C RHS Outer surface: Near Violet-Blue group 93B RHS Color: Lower Lip: When opening: Inner surface: Near Violet-Blue group 93A RHS Outer surface: Near Violet-Blue group N92B RHS Fully opened: Inner surface: Near Violet-Blue group 93C RHS Outer surface: Near Violet-Blue group 93B RHS Aging: Inner surface: Near Violet-Blue group 93D RHS Outer surface: Near Violet-Blue group 93C RHS.
   Color.—Tube: When opening: Inner surface: Near Violet-Blue group 91D RHS Outer surface: Near Violet-Blue group 91A RHS Fully opened: Inner surface: Near Violet-Blue group 92A RHS Outer surface: Near Violet-Blue group 92C RHS Fading: Inner surface: Near Violet-Blue group 91D RHS Outer surface: Near Violet-Blue group 94B RHS.
   Bud.—Shape: Ovoid Length: 0.5 CM Diameter: 0.2 CM Color: Near Violet—Blue group 93A RHS.
   Sepals.—Shape: Quantity per flower: 2 lobes fused in base to form a tub Length: Approximately 0.6 cm Width: Approximately 0.3 cm Margin: Flat Texture: Velvety, with rough lines longwise, pulverulent Color: Near Violet-Blue group 93A RHS.
Reproductive organs:
   Stamens.—Number: 2 Filament length: Approximately 0.1 cm.
   Anthers.—Shape: Obvuate Length: Approximately 0.1 CM Color: Near Greyed-Purple group N187 RHS Pollen: Color: Near Yellow group 14A RHS Quantity: Medium.
   Pistil.—Number: 1 Length: Approximately 1.2 cm Style: Length: Approximately 1 cm Color: Near Violet group 85D RHS.
   Stigma.—Shape: V shape with one side longer than the other Color: Near Violet group 85B RHS.
   Ovary color.—Near Green-Yellow group 1D RHS.
Other characteristics:
   Seeds and fruits.—1-4 seeds per flower, ovoid shape. Dark Grey-Brown in color, not accurately measured with R.H.S. chart.
   Disease/pest resistance.—Neither resistance nor susceptibility to normal diseases and pests of Salvia farinacea has been observed. Pests which can be found in Salvia include aphids and thrips. Typical diseases are Peronospora lamii and Erysiphe cichoracearum.

What is claimed is:

1. A new and distinct cultivar of Salvia plant named ‘DSALDPOCN’ as herein illustrated and described.
